(19) United States
(12) Patent Application Publication
(10) Pub. No.: US 2018/0110741 A1
(43) Pub. Date: Apr. 26, 2018

(54) ASTAXANTHIN COMPOSITIONS (I)

(71) Applicant: BASF SE, Ludwigshafen (DE)

(72) Inventors: JESPER FELDTHUSEN JENSEN, Nieder-Olm (DE); Tamara Alexandra RAUSCH, Viernheim (DE); Claudia SCHOLTEN, Mannheim (DE); Bernd SCHÄFER, Dierbach (DE)

(21) Appl. No.: 15/559,306

(22) PCT Filed: Mar. 18, 2016
(86) PCT No.: PCT/EP2016/055919

§ 371 Date: Sep. 18, 2017

(30) Foreign Application Priority Data
Mar. 19, 2015 (EP) 15159828.1

(51) Int. Cl. A61K 31/122; A23K 20/105; A23K 20/179; A23L 5/43; A23L 33/105; A61P 19/02; A61P 27/02; A61P 17/00; A61P 9/00; A61P 35/00; A61P 31/04; A61P 3/10; A61P 25/00; A61P 15/08; A61P 37/00

(52) U.S. Cl. CPC A61K 31/122; A23V 2002/00; A23K 20/179; A23L 5/43; A23L 33/105; A61P 19/02; A61P 27/02; A61P 17/00; A61P 9/00; A61P 35/00; A61P 31/04; A61P 3/10; A61P 25/00; A61P 15/08; A61P 37/00; A23K 20/105

(57) ABSTRACT

The present invention relates to astaxanthin compositions for use in foodstuffs, food supplements or feedstuffs or as a medicament.

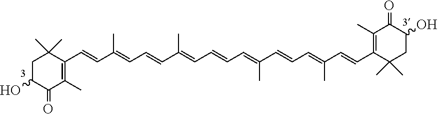 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

Astaxanthin compositions which comprise up to at least 50% by weight, in particular at least 60% by weight, particularly at least 70% by weight, specifically at least 80% by weight or at least 90% by weight, based on the total weight of astaxanthin and astaxanthin derivatives in the composition, of an astaxanthin monoester with an aliphatic, unbranched C₁₀C₂₂-monocarboxylic acid have a particularly good bioavailability when at least 90% by weight of the monoester present in the astaxanthin composition is a monoester with precisely one aliphatic, unbranched C₁₀-C₂₂-monocarboxylic acid. They are therefore particularly suitable for the use in foodstuffs, food supplements or feedstuffs and for the therapeutic use as medicament and as constituent for medicinal preparations and pharmaceutical compositions.

### Astaxanthin Compositions (I)

The present invention relates to astaxanthin compositions for use in foodstuffs, food supplements or feedstuffs or as a medicament.

Astaxanthin (3,3′-dihydroxy-β,β-carotene-4,4′-dione) is a red carotenoid colorant from the group of xanthophylls, which is described by the formula hereinbelow (what is shown is the all-trans isomer).

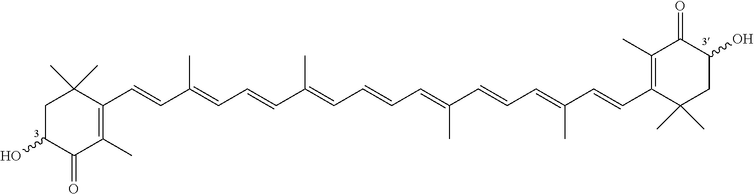 [SMILES: CC1=C(/C=C/C(C)=C/C=C/C(C)=C/C=C/C=C(C)/C=C/C=C(C)/C=C/C2=C(C)C(=O)C(O)CC2(C)C)C(C)(C)CC(O)C1=O]

Astaxanthin, hereinbelow also AXT, has an asymmetric center in the 3 and 3′ position and can therefore exist as a diastereomer mixture of the (3R, 3′R), (3S, 3′S) and (3S, 3′R) isomers, as a racemate of the (3R, 3′R) and of the (3S, 3′S) isomer or in the form of the pure isomers. Synthetic AXT is strictly a mixture of the diastereomers (3S, 3′S), (3R,3′S) and (3R, 3′R). AXT which has been obtained from natural sources may, depending on the respective natural source, be present in the virtually pure (3S, 3′S) or (3R, 3′R) form. Likewise, enantiomerically pure astaxanthin is obtainable by total synthesis.

Astaxanthin is predominantly prepared synthetically—see in this context G. Britton, S. Liaanen-Jensen, H. Pfander (editor), Carotenoids, Vol. 2, Birkhauser Verlag, Basel, 1996, in particular p. 11, p. 267 et seq. and p. 281 et seq. and literature cited therein; B. Schafer, Naturstoffe der chemischen Industrie [Natural substances of the chemical industry], Akademischer Verlag, Heidelberg, 2007, p.427 et seq. and literature cited therein; EP 1197483, EP 1285912.

Besides the synthetic astaxanthin preparation, astaxanthin can also be obtained on an industrial scale from Haematococcus pluvialis (E. A. Johnson, G. H. An, Crit. Rev. Biotechnol. 1991, 11: 297-326). This gives a complex mixture of mono- and di-fatty acid esters of astaxanthin—see in this context also D. Breithaupt, J. Agric. Food. Chem. 2004, 52: 3870-3875.

Astaxanthin is approved as a feed additive and is predominantly used as a feed component for various animals, in particular for salmon and trout. Thus, AXT has a vitamin-like effect, therefore has a positive effect on the fertility and immune response of fish in fish farms and results in an appealing pigmentation of the flesh. Astaxanthin is also used as an additive for food supplements, as a cosmetic additive with antioxidant properties or as a colorant for foodstuffs. AXT is capable of protecting the skin from stress caused by UV radiation, in which function it has a considerably more powerful activity than vitamin E. AXT supplements the protective effect of sun protectants and cannot be washed off.

It has recently been reported that AXT is suitable in principle for the treatment of a series of various health disorders—see, for example, J. H. Weisburger, Am. J. Clin. Nutr. 1991, 53: 226S-237S; M. Guerin, Trends in Biotechnology, 2003, 21: 210-216; Ghazi Hussein et al., J. Nat. Prod., 2006, 69: 443-449, Astaxanthin and Human Health-Literature Survey (Algatechnologies): http://www.astapure-algatech.com/Data/Sub2_1/Files/algatechnologies.pdf in each case with further detections.

Thus, in-vivo studies on animal models allow the assumption that AXT lowers the blood sugar level and improves various parameters of metabolic syndrome (K. Uchiyama, Redox. Rep. 2000, 7(5): 290-3; G. Hussein et al., J. Pharm. Sci. 2007, 100(Suppl. 1):176p; M. Ikeuchi et al. Biosci. Biotechnol. Biochem. 2007, 71:893-9; Naito, Y., K. Uchiyama, et al. Biofactors 2004, 20(1): 49-59).

In blood hypertension models, AXT results in increased blood flow and vascular tone—see, for example, H. Yanai et. al. (2008) Integrated Blood Pressure Control 1:1-3.

Epidemiological studies suggest that carotenoids such as astaxanthin have positive effects on heart diseases—D. A. Cooper et al., Nutr. Rev. 1999, 57: 210-214, J. Krappi et. al., Int. J. Nutr. Res. 2006, 77(1): 3-11. Owing to its antioxidant effect, in particular its effect of inhibiting the oxidation of low-density lipoproteins, carotenoids such as astaxanthin have been proposed for preventing arteriosclerosis—see T. Iwamoto et al., J. Atheroscler. Thromb. 2000, 7: 216-222.

Furthermore, various sources have reported that the administration of AXT reduces the risk of cancer—see J. H. Weisburger, Am. J. Clin. Nutr. 1991, 53: 226S - 237S. In-vivo studies in mice demonstrate that astaxanthin slows down or indeed inhibits tumor growth, in particular of mammary carcinomas, bladder carcinomas and lung carcinoma—see B. P. Chew et al., Anticancer Res. 1999, 19: 1849-1853, T. Tanaka et al. Carcinogenesis 1994, 15: 15-19 and D. A. Cooper et al., Nutr. Rev. 1999, 57: 133-145. Moreover, AXT appears to promote the formation of connexin 43 and therefore has a chemoprotective effect against other cancer diseases (see A. L. Vine et al., Nutr. Cancer 52(1) (2005), 105-113).

Moreover, astaxanthin plays a role in modulating the immune system—see H. Jyonouchi et al. J. Nutr. 1995, 125: 1570-1573 —, and has an antiinflammatory effect; J. Park, et al. Nutr. Metab. 2010, 7: 18-27, Kurashige et al. Physiol. Chem. Phys. Med. 1990, NMR22:27-38, K. Ohgami et al. Investigative Ophthalmology & Visual Sci. 2003, 44(6):2694-2701 and Y. Suzuki et al. Exp. Eye Res. 2006, 82(2): 275-281. It has also been proposed for the treatment of autoimmune diseases such as Crohn's disease.

Furthermore, astaxanthin has been proposed for the treatment of joint diseases, in particular for improving the symptoms of inflammatory joint diseases—see Y. Nir, G. Spiller C. Multz C. Effect of an Astaxanthin-containing product on carpal tunnel syndrome. American College of Nutrition Annual Meeting, San Antonio, Tex., October 2002, Y. Nir, G. Spiller. Los Altos, Calif.: Health Research and Studies Center 2002. Y. Nir, G. Spiller. J Am Coll Nutr, 2002, 21.

It has been demonstrated in animal models that the administration of AXT has an advantageous effect on the course of bacterial infections, in particular inflammations of the gastrointestinal tract, caused by infection with Helicobacter pylori—see M. Bennedsen et al., lmmunol. Lett. 1999, 70: 185-189.

Furthermore, astaxanthin has been proposed for preventing eye diseases such as damage of the lens and of the retina by UV radiation, macular degeneration, cataract and glaucoma, for improving male fertility, and for improving memory, concentration and physical fitness—K. Sawaki et al. Journal of Clinical Therapeutics & Medicines, 2002, 18: 73 -88.

Astaxanthin can contribute substantially to delaying skin aging and reducing the development of wrinkles, age spots and freckles—Suganuma K et al., Jichi Medical University Journal. 2012, 35:25-33; K. Tominaga K et al., Acta Biochim Pol. 2012; 59(1):43-7; E. Yamashita, Carotenoid Science. 2006; 10: 91-5; A. Satoh et al. Oyo Yakuri Pharmacometrics, 2011, 80 (1/2): 7-11.

Astaxanthin is therefore, in principle, of interest for the preparation of medicaments and for feedstuffs, foodstuffs and food supplements, in particular those for human nutrition. However, the main problem is that astaxanthin, when administered orally, is poorly absorbed by the human or animal organism and the attained serum levels in blood serum or lymph serum are only low—see, for example, R. M. Clark et al. Lipids 2000, 35: 803-806; J. Mercke Odeberg et al., Europ. J. Pharm. Sci. 2003, 19: 299-304, M. Osterlie et al., J. Nutr. Biochem, 2000, 11: 482-490. Another problem is the fact that astaxanthin is sparingly soluble in the adjuvants which are suitable for the preparation of the administration of food supplements and medicaments. While this problem can be overcome in part by esterifying astaxanthin with fatty acids, the diesters of astaxanthin with fatty acids surprisingly have comparable properties in respect of the serum levels attainable as nonesterified astaxanthin—see D. A. White et al., Aquacult. Res. 2002, 33: 343-350, Odeberg et al., Eur. J. Pharmac. Sciences 2003, 19: 299-304, Miyazawa et al., Biosci Biotechnol Biochem, 2011, 76: 1856-1858, Osterlie et al., J. Nutr. Biochem., 11, 2000: 482-490, Okada et al., Biosci. Biotechnol. Biochem., 2009, 73: 1928-32, Coral-Hinostroza et al., Comparative Biochemistry and Physiology, Part C, 139, 2004: 99-110.

It is known that astaxanthin is absorbed by passive diffusion into the intestinal epithelium for which a certain amount of fat is required and which can therefore be promoted by fats in the food (see G. N. Coral-Hinostroza et al. Comp. Biochem. Physiol. Part C 2004, 139: 99-110 with further detections). J. Mercke Odeberg et al., Europ. J. Pharm. Sci. 2003, 19: 299-304, describe that the oral bioavailability of astaxanthin can be improved by special fat-based formulations, with the emulsifiers used for this purpose having a great effect on the bioavailability. The improvement of the oral bioavailability by formulations is, however, limited, which restricts the possible uses of astaxanthin.

The applicant's own studies have demonstrated that “natural astaxanthin” obtained from Haematococcus pluvialis, a complex mixture of astaxanthin fatty acid esters, has an oral bioavailability which is improved over astaxanthin or di-fatty acid esters of astaxanthin. However, the commercial availability of natural astaxanthin is very limited. Owing to the complexity of such a mixture, it is of little interest for pharmaceutical applications.

It is therefore an object of the present invention to overcome the disadvantages of the prior art and to prepare in particular astaxanthin derivatives or astaxanthin compositions which firstly have better oral bioavailability and which are furthermore more readily available than “natural astaxanthin” obtained from Haematococcus pluvialis.

The earlier European patent application 14184483.7 describes the preparation of di-fatty acid esters of astaxanthin by esterifying astaxanthin with fatty acid chloride in the presence of a tertiary amine base. If the free acid is employed instead of the acid chloride, the predominant product is the mono-fatty acid ester which can be obtained from the reaction mixture by removing unreacted astaxanthin. Moreover, it is possible to obtain the mono-fatty acid esters by employing substoichiometric amounts of acid chloride in the mixture with unreacted astaxanthin and di-fatty acid ester. The monoester can be isolated from the mixture, for example by chromatography.

Surprisingly, it has now been found that astaxanthin compositions which comprise at least 50% by weight, in particular at least 60% by weight, especially at least 70% by weight, specifically at least 80% by weight or at least 90% by weight, based on the total weight of astaxanthin and astaxanthin derivatives in the composition, of a monoester of astaxanthin with an aliphatic, unbranched C₁₀-C₂₂-monocarboxylic acid display a particularly good bioavailability when at least 90% by weight of the monoester which is present in the astaxanthin composition is a monoester with precisely one aliphatic, unbranched C₁₀-C₂₂-monocarboxylic acid.

Subject-matter of the invention is therefore the use of astaxanthin compositions in foodstuffs, food supplements or feedstuffs, where the astaxanthin composition comprises at least 50% by weight, in particular at least 60% by weight, especially preferably at least 70% by weight, specifically at least 80% by weight or at least 90% by weight, based on the total weight of astaxanthin and astaxanthin derivatives in the composition, of a monoester of astaxanthin with an aliphatic, unbranched C₁₀-C₂₂-monocarboxylic acid, where at least 90% by weight of the monoester present in the astaxanthin composition is a monoester with precisely one aliphatic, unbranched C₁₀-C₂₂-monocarboxylic acid.

Subject-matter of the invention are also the astaxanthin compositions described here and hereinbelow for the therapeutic use as medicament and as constituent for medicinal preparations or pharmaceutical products. Accordingly, the invention also relates to a medicament which comprises an astaxanthin composition according to the invention and at least one auxiliary or adjuvant which is suitable for pharmaceutical purposes.

The present invention also relates to additives for foodstuffs which comprise the astaxanthin composition according to the invention and at least one additive which is suitable for foodstuffs, i.e. at least one adjuvant which is suitable for foodstuffs.

The present invention also relates to additives for feedstuffs which comprise the astaxanthin composition according to the invention and at least one additive which is suitable for feedstuffs, i.e. at least one adjuvant which is suitable for feedstuffs.

The present invention also relates to food supplements which comprise the astaxanthin composition according to the invention and at least one additive which is suitable for food supplements, i.e. at least one adjuvant which is suitable for food supplements.

Naturally, the abovementioned food additives, feed additives, food supplements and pharmaceutical composition comprise the astaxanthin composition according to the invention as the sole astaxanthin-comprising component, and they comprise in particular no further, nonesterified astaxanthin or further astaxanthin derivatives.

The invention entails a series of advantages. Firstly, the compositions according to the invention display a better oral bioavailability than nonesterified astaxanthin or corresponding diesters, and the astaxanthin serum levels achieved in the blood plasma are higher by a multiple. Surprisingly, the oral bioavailability of the astaxanthin compositions according to the invention is even better than that of “natural astaxanthin”. Moreover, the compositions can readily be synthesized. Last, but not least, the compositions according to the invention are also particularly attractive for pharmaceutical applications, owing to the fact that they are less complex in comparison with “natural astaxanthin” and that their oral bioavailability is improved.

In particular, the astaxanthin compositions according to the invention are suitable for preventing and treating a multiplicity of disease symptoms and health disorders, such as, for example for
prevention and treatment of joint diseases, in particular inflammatory joint diseases such as rheumatoid arthritis, arthrosis and carpal tunnel syndrome,
prevention and treatment of eye diseases such as damage to the lens or the retina, for example for the prevention of macular degeneration, cataract or glaucoma and for the protection of the lens and retina against damage by UV radiation,
prevention and treatment of skin diseases and for the prevention and reduction of skin aging, in particular for the prevention and reduction of wrinkles, age spots and freckles,
prevention and treatment of heart diseases, in particular for the prevention of myocardial infarction and for improving reconvalescing after myocardial infarction,
prevention and treatment of type 2 diabetes and associated concomitant diseases, for example prevention and treatment of metabolic syndrome, for lowering the blood sugar level in insulin-resistant individuals or for the treatment and prevention of diabetic nephropathy,
prevention and treatment of tumor diseases, in particular cervical carcinoma, bladder carcinoma, mammary carcinoma and lung carcinoma,
prevention and treatment of diseases of the blood vessels such as arteriosclerosis,
treatment of bacterial infections, in particular for improving the course of diseases of the gastrointestinal tract which are caused by infection with Helicobacter pylori,
prevention and treatment of inflammatory diseases, in particular of autoimmune diseases such as Crohn's disease,
prevention and treatment of degenerative nerve disorders, such as Parkinson's disease,
for improving male fertility,
for improving the general state of health,
for improving memory and concentration ability,
for improving physical fitness,
for strengthening the immune system.

A treatment within the meaning of the invention comprises not only the treatment of acute or chronic signs, symptoms and/or malfunctions, but also a preventative treatment (prophylaxis), in particular as relapse prophylaxis or phase prophylaxis. The treatment can be directed toward being symptomatic, for example as suppression of symptoms. It may take place short-term, be directed toward being medium-term, or it can also be a long-term treatment, for example as part of a maintenance therapy.

Herein and hereinbelow, a feedstuff is understood as being a product for the nutrition of nonhuman beings, in particular of nonhuman mammals, but also of fish. A feed additive, accordingly, is understood as meaning a formulation, for example in the form of a powder or a liquid, which can be incorporated into the feedstuff during its manufacture.

Herein and hereinbelow, a foodstuff is understood as meaning a nutrient-containing product for the nutrition of humans, which serves primarily for the intake of nutrients such as fats, proteins and carbohydrates. A food additive, accordingly, is understood as meaning a formulation, for example in the form of a powder or a liquid, which can be incorporated into the foodstuff during its manufacture.

Herein and hereinbelow, a food supplement is understood as meaning a formulation which is suitable for oral administration and which is ingested by humans in addition to the traditional food, with the primary aim of administering active substances, in the present case astaxanthin or a derivative, in order to achieve, in this manner, an improvement of the general diet or the general state of health. Food supplements occupy the borderline between foodstuffs and medicaments and, in EU law, are regulated by Guideline 2002/46/EC.

The prefix Cₙ-Cₘ used herein and hereinbelow in generic terms such as monocarboxylic acid or fatty acid indicates a range for the number of carbon atoms which a member of the group to which the generic term refers may include.

The astaxanthin compositions according to the invention comprise predominantly, i.e. to at least 50% by weight, in particular at least 60% by weight, especially preferably at least 70% by weight, specifically at least 80% by weight or at least 90% by weight, based on the total weight of astaxanthin and astaxanthin derivatives in the composition, a monoester of astaxanthin with an aliphatic, unbranched C₁₀-C₂₂-monocarboxylic acid.

Besides this, the astaxanthin compositions according to the invention may also comprise nonesterified astaxanthin and/or one or more astaxanthin diesters, whose content does not exceed, in accordance with the invention, 50% by weight, in particular 40% by weight, especially preferably 30% by weight, specifically 20% by weight or 10% by weight, based on the total weight of astaxanthin and astaxanthin derivatives in the composition. The astaxanthin composition however may also be composed exclusively or almost exclusively, i.e. to more than 95% by weight, of the monoester of astaxanthin with the respective aliphatic, unbranched C₁₀-C₂₂-monocarboxylic acid.

In accordance with the invention, the monoester is uniform, i.e. it is essentially, i.e. to at least 90% by weight, the monoester of precisely one aliphatic, unbranched C₁₀-C₂₂-monocarboxylic acid. In this context, the term “precisely one” means that the monoester is the monoester of a specific, aliphatic, unbranched C₁₀-C₂₂-monocarboxylic acid, i.e. an aliphatic, unbranched C₁₀-C₂₂-monocarboxylic acid with the same empirical formula. In the case of the unsaturated, aliphatic, unbranched C₁₀-C₂₂-monocarboxylic acids, a specific aliphatic C₁₀-C₂₂-monocarboxylic acid comprises not only the mixtures of the various double-bond isomers, but also the cis- and trans-isomer mixtures. If the composition also comprises astaxanthin diesters, the diesters are, as a rule, the diester with the aliphatic, unbranched C₁₀-C₂₂-monocarboxylic acid which also forms the monoester.

The aliphatic, unbranched C₁₀-C₂₂-monocarboxylic acids can be saturated or unsaturated. Unsaturated, aliphatic, unbranched C₁₀-C₂₂-monocarboxylic acids can include one or more, for example 1, 2, 3, 4, 5 or 6, ethylenic double bonds. The latter may be in cis- or trans-configuration in respect of the respective double bond. The aliphatic, unbranched C₁₀-C₂₂-monocarboxylic acids are typically nonfunctionalized, i.e. they include no further heteroatoms in addition to the two oxygen atoms of the carboxyl group.

Examples of saturated, aliphatic, unbranched C₁₀-C₂₂-monocarboxylic acids are predominantly even-numbered, saturated C₁₀-C₂₂-monocarboxylic acids such as capric acid, lauric acid, myristic acid, palmitic acid, stearic acid, arachic acid and behenic acid,

Examples of unsaturated, aliphatic, unbranched C₁₀-C₂₂-monocarboxylic acids are mainly
monounsaturated C₁₀-C₂₂-monocarboxylic acids such as myristoleic acid, palmitoleic acid, oleic acid and its double-bond isomers petroselic acid, elaidic acid and vaccenic acid (C18-1 acids), C20-1 acids such as eicosenic acid and gadoleic acid, C22-1 acids such as cetoleic acid and erucic acid.
Polyunsaturated C₁₀-C₂₂-monocarboxylic acids such as linoleic acid (C18-2 acid), the C18-3 acids alpha-linolenic acid, gamma-linolenic acid, calendulic acid, punicic acid, alpha-elaeostearic acid and beta-elaeostearic acid, the C20-4 acid arachidonic acid, the C20-5 acids timnodonic acid (=eicosapentaenoic acid) and clupanodonic acid, the C22-5 acid docosapentaenoic acid and the C22-6 acid cervonic acid.

Preferred C₁₀-C₂₂-monocarboxylic acids are selected from among saturated and unsaturated, aliphatic, unbranched C₁₆-C₂₂-monocarboxylic acids.

Preferred C₁₀-C₂₂-monocarboxylic acids are selected from among mono- or polyunsaturated, aliphatic, unbranched C₁₀-C₂₂-monocarboxylic acids, in particular among mono- or polyunsaturated, aliphatic, unbranched C₁₆-C₂₂-monocarboxylic acids. The at least monounsaturated C₁₀-C₂₂-monocarboxylic acid is selected in particular from among C18-1, C18-2, C18-3 and C18-4 fatty acids. Specifically, it is a C18-1 fatty acid.

Preferred among the astaxanthin compositions according to the invention are those in which at least 90% by weight of the astaxanthin monoester is a monoester of astaxanthin with precisely one aliphatic, unbranched C₁₆-C₂₂-monocarboxylic acid.

Preferred among the astaxanthin compositions according to the invention are furthermore those in which at least 90% by weight of the astaxanthin monoester is a monoester of astaxanthin with precisely one aliphatic, unbranched C₁₀-C₂₂-monocarboxylic acid which is mono- or polyunsaturated, including monoesters of cis/trans mixtures of the unsaturated, aliphatic, unbranched C₁₀-C₂₂-monocarboxylic acid and mixtures of the double-bond isomers of this unsaturated, aliphatic, unbranched C₁₀-C₂₂-monocarboxylic acid.

Among the astaxanthin compositions according to the invention, others which are also preferred are those in which at least 90% by weight of the astaxanthin monoester is a monoester of astaxanthin with precisely one aliphatic, unbranched C₁₆-C₂₂-monocarboxylic acid which is mono- or polyunsaturated, including monoesters of cis/trans mixtures of the unsaturated, aliphatic, unbranched C₁₆-C₂₂-monocarboxylic acid and mixtures of the double-bond isomers of this unsaturated, aliphatic, unbranched C₁₆-C₂₂-monocarboxylic acid. In this context, it has proved as particularly advantageous for not more than 60%, in particular not more than 40% and specifically not more than 30% of the unsaturated fatty acids to be present as trans-fatty acids, i.e. not more than 60%, in particular not more than 40% and specifically not more than 30% of all double bonds in the fatty acid residue are present in the trans form.

Among the astaxanthin compositions according to the invention, others which are in particular preferred are those in which at least 90% by weight of the astaxanthin monoester is a monoester of astaxanthin with an unbranched fatty acid which is selected from among C18-1, C18-2, C18-3 and C18-4 fatty acids. In this context, it has proved particularly advantageous for not more than 60%, in particular not more than 40% and specifically not more than 30% of the C18-1, C18-2, C18-3 and C18-4 fatty acids to be present as trans-fatty acids, i.e. not more than 60%, in particular not more than 40% and specifically not more than 30% of all double bonds in the fatty acid residue are present in the trans form.

Among the astaxanthin compositions according to the invention, others which are in particular also preferred are those in which at least 90% by weight of the astaxanthin monoester is a monoester of astaxanthin with an unbranched fatty acid which is selected from among C16-0 and C18-0 fatty acids.

Among the astaxanthin compositions according to the invention, others which are in particular preferred are those in which at least 90% by weight of the astaxanthin monoester is a monoester of astaxanthin with C18-1 fatty acid, in particular oleic acid or the cis/trans isomer mixture of oleic acid. In these monoesters of the C18-1 fatty acid, it is preferred for not more than 60%, in particular not more than 40% and specifically not more than 30% of the double bonds in the C18-1 fatty acid residue to be present in the trans form.

In a group of embodiments, at least 40% by weight, in particular at least 60% by weight or at least 80% by weight of the monoester, in particular of the monoester of the unsaturated, aliphatic, unbranched C₁₆-C₂₂-monocarboxylic acid, especially preferably of the monoester of C18-1, C18-2, C18-3 and C18-4 fatty acid and specifically of the monoester of oleic acid, are present as the monoester of all-E astaxanthin.

In a further group of embodiments, 20 to 90% by weight, in particular 30 to 80% by weight or 40 to 70% by weight of the monoester, in particular of the monoester of the unsaturated, aliphatic, unbranched C₁₆-C₂₂-monocarboxylic acid, especially preferably of the monoester of C18-1, C18-2, C18-3 and C18-4 fatty acid and specifically of the monoester of oleic acid, are present as the monoester of all-E astaxanthin and 10 to 80% by weight, in particular 20 to 70% by weight or 30 to 60% by weight are present as one or more Z isomers of astaxanthin, for example as 9-Z, 13-Z or 15-Z isomer or as a mixture of two or three of these Z isomers of astaxanthin.

In a further group of embodiments, at least 40% by weight, in particular at least 60% by weight or at least 80% by weight of the monoester of a saturated, aliphatic, unbranched C₁₆-C₂₂-monocarboxylic acid, especially preferably of the monoester of C16-0 or C18-0 fatty acid, are present as the monoester of all-E astaxanthin.

In a further group of embodiments, 20 to 90% by weight, in particular 30 to 80% by weight or 40 to 70% by weight of the monoester of a saturated, aliphatic, unbranched C₁₆-C₂₂-monocarboxylic acid, especially preferably of the monoester of C16-0 or, C18-0 fatty acid, are present as the monoester of all-E astaxanthin and 10 to 80% by weight, in particular 20 to 70% by weight or 30 to 60% by weight of the monoester of this fatty acid are present as one or more Z isomers of astaxanthin, for example as 9-Z, 13-Z or 15-Z isomer or as a mixture of two or three of these Z isomers of astaxanthin.

In a further group of embodiments, at least 80% by weight, in particular at least 90% by weight of the monoester, in particular of the monoester of the unsaturated, aliphatic, unbranched C₁₆-C₂₂-monocarboxylic acid, especially preferably of the monoester of C16-0, C18-0, C18-1, C18-2, C18-3 and C18-4 fatty acid and specifically of the monoester of oleic acid, are present as the monoester of the 3S, 3′S enantiomer of astaxanthin.

In a further group of embodiments, at least 80% by weight, in particular at least 90% by weight of the monoester, in particular of the monoester of the unsaturated, aliphatic, unbranched C₁₆-C₂₂-monocarboxylic acid, especially preferably of the monoester of C16-0, C18-0, C18-1, C18-2, C18-3 and C18-4 fatty acid and specifically of the monoester of oleic acid, are present as the monoester of the 3R, 3′R enantiomer of astaxanthin.

In a further group of embodiments, the monoester, in particular the monoester of the unsaturated, aliphatic, unbranched C₁₆-C₂₂-monocarboxylic acid, especially preferably the monoester of C16-0, C18-0, C18-1, C18-2, C18-3 and C18-4 fatty acid and specifically the monoester of oleic acid, is the monoester of a mixture of the 3S, 3′S enantiomer, of the 3R, 3′S-meso form and of the 3R, 3′R enantiomer of astaxanthin, in particular the statistic mixture of these astaxanthin forms.

In accordance with one group of embodiments of the invention, the astaxanthin compositions according to the invention or the corresponding monoesters are employed in food additives or feed additives. These food or feed additives comprise the astaxanthin composition according to the invention and at least one auxiliary or adjuvant which is suitable for foodstuffs or feedstuffs. An adjuvant which is suitable for foodstuffs or feedstuffs is to be understood as meaning for the purposes of the invention a substance which is approved for animal and/or human nutrition.

Suitable adjuvants are in particular those which permit the additive to be incorporated into foodstuffs by diluting the astaxanthin composition in the additive to a better manageable concentration. These include above all carriers, in particular liquid carriers such as vegetable oils and organic solvents, but also solid carriers such as fats, fatty acids, waxes, fatty alcohols and fatty acid esters of fatty alcohols, carbohydrates, sugar alcohols and inorganic fillers which are approved for the manufacture of foodstuffs or feedstuffs.

Examples of solvents are:
C₁-C₆-alkanols such as, for example, ethanol, 1-propanol, 2-propanol, 1-butanol, 2-butanol, 2-methyl-1-propanol, 3-methyl-1-butanol, 1-pentanol and their mixtures;
C₁-C₄-alkyl esters of aliphatic C₁-C₄-carboxylic acids such as, for example, the methyl, ethyl, n-propyl, isopropyl, n-butyl, 2-butyl or isobutyl esters of formic acid, acetic acid, propionic acid or butyric acid, such as methyl acetate, ethyl acetate, n-propyl acetate, isopropyl acetate, n-butyl acetate, isobutyl acetate, ethyl formate and their mixtures; and
aliphatic, in particular noncyclic, ketones having 3 to 6 C atoms such as acetone, methyl ethyl ketone, isobutyl methyl ketone and their mixtures; and
mixtures of the abovementioned solvents from various classes of the abovementioned solvents.

Suitable carbohydrates include mono-, di-, oligo- and polysaccharides. Examples of monosaccharides and disaccharides are mainly glucose, fructose, galactose, mannose, maltose, sucrose and lactose. Suitable polysaccharides are starch and oligomeric starch degradation products (dextrins) and cellulose powder.

Suitable sugar alcohols are mainly sorbitan and glycerol,

Suitable fats and oils may be of synthetic, mineral, vegetable or animal origin.

Examples of oils are vegetable oils such as soya oil, sunflower oil, corn oil, linseed oil, rapeseed oil, safflower oil, wheat germ oil, rice oil, coconut oil, almond oil, apricot kernel oil, palm oil, palm kernel oil, avocado oil, jojoba oil, hazelnut oil, walnut oil, peanut oil, pistachio oil, triglycerides of medium-chain (=C₈-C₁₀) vegetable fatty acids (so-called MCT oils) and PUFA oils (PUFA=polyunsaturated fatty acids such as eicosapentaenoic acid (EPA), docosahexaenoic acid (DHA) and α-linolenic acid), furthermore semisynthetic triglycerides, for example caprylic/capric acid triglycerides such as the miglyol types, furthermore liquid paraffin, liquid hydrogenated polyisobutenes, squalane, squalene, furthermore animal oils and fats such as fish oils including mackerel oil, sprat oil, tuna oil, halibut oil, cod oil and salmon oil. Preferred are vegetable oils and oils of animal origin which are liquid at 40° C., in particular vegetable oils such as soya oil, sunflower oil, safflower oil, corn oil, olive oil, linseed oil, rapeseed oil, rice oil, coconut oil, peanut oil, palm oil, palm kernel oil, PUFA oils, MCI oils, furthermore fish oils, and mixtures of these oils.

In contrast to oils, fats usually have a melting point of above 30° C. Examples of fats are:
saturated fatty acids having 12 to 30 C atoms (saturated C₁₂-C₃₀-fatty acids), in particular 14 to 28 C atoms (saturated C₁₄-C₂₈-fatty acids) and specifically 16 to 24 C atoms (saturated C₁₆-C₂₄-fatty acids) such as myristic acid, palmitic acid, margaric acid, stearic acid, arachic acid, behenic acid, cerotic acid, melissic acid and lignoceric acid, and their mixtures;
fatty acid esters of saturated fatty acids having 14 to 30 C atoms, in particular 14 to 28 C atoms and specifically 16 to 24 C atoms, such as the esters of palmitic acid, margaric acid, stearic acid, arachic acid, behenic acid, cerotic acid, melissic acid _{—} and lignoceric acid and their mixtures, in particular the mono-, di- and triglycerides of the abovementioned fatty acids and their mixtures (hereinbelow referred to as fatty acid glycerides), in particular of the abovementioned saturated fatty acids and specifically those saturated fatty acids which have 14 to 28 C atoms and specifically 16 to 28 C atoms, and esters of the abovementioned saturated fatty acids having preferably 14 to 28 C atoms and specifically 16 to 28 C atoms, with C₁₀-C₃₀-fatty alcohols, in particular with C₁₄-C₂₈-fatty alcohols. The abovementioned esters of saturated fatty acids may also comprise mono- or polyunsaturated fatty acids in esterified form in an amount of up to 10% by weight, based on the fatty acid content in the ester. In particular, the content of unsaturated fatty acid components in these esters amounts to less than 5% by weight, based on the total fatty acid components in the ester.

The oils and fats may be refined oils or crude oils/fats which still comprise origin-specific impurities such as proteins, phosphate, alkali metal salts, alkaline earth metal salts and the like in usual amounts.

Suitable fatty alcohols are in particular saturated, aliphatic alcohols having 8 to 30 C atoms (hereinbelow also C₈-C₃₀-fatty alcohols), such as, for example, cetyl alcohol, stearyl alcohol, nonadecanol, arachidyl alcohol, behenyl alcohol, lignoceryl alcohol, ceryl alcohol, myricyl alcohol and melissyl alcohol.

Suitable waxes are in particular natural waxes of vegetable or animal origin such as beeswax, candelilla wax, shellac wax, shea butter and carnauba wax, carbohydrate waxes such as paraffin waxes, ceresin, Sasol waxes, ozokerite and microwaxes.

Examples of inert inorganic fillers which are suitable for foodstuffs are inorganic materials in pulverulent form (inorganic fillers), for example oxides such as aluminum oxide, silica, titanium dioxide, silicates such as sodium silicate, magnesium silicate, talc, calcium silicate, zinc silicate, aluminum silicates such as sodium aluminum silicate, potassium aluminum silicate, calcium aluminum silicate, bentonite, kaolin and sodium chloride.

The adjuvants which are suitable for foodstuffs and feedstuffs furthermore include dispersants, including lipophilic dispersants for dispersing the astaxanthin compositions in lipophilic carriers and protective colloids for dispersing the astaxanthin compositions according to the invention in hydrophilic carriers such as water, furthermore antioxidants (oxidation stabilizers), and colorants which are approved for foodstuffs.

Examples of antioxidants are tocopherols such as α-tocopherol, α-tocopherol palmitate, α-tocopherol acetate, tert-butyl hydroxytoluene, tert-butylhydroxyanisole, ascorbic acid, its salts and esters such as, for example, sodium ascorbate, calcium ascorbate, ascorbyl phosphate ester and ascorbyl palmitate and ethoxyquin. If desired, the antioxidants are typically present in the additives according to the invention in amounts of from 0.01 to 10% by weight, based on the total weight of the additive.

Examples of preservatives include benzoic acid and its salts, in particular its sodium, potassium and calcium salts, 4-hydroxybenzoic acid (PHB) and its salts, in particular its sodium, potassium and calcium salts, the salts of the PHB alkyl esters, such as the sodium salt of the PHB methyl ester, the sodium salt of the PHB ethyl ester and the sodium salt of the PHB propyl ester, sorbic acid and its salts, in particular its sodium, potassium and calcium salts, salts of propionic acid such as, in particular, its sodium, potassium and calcium salts, boric acid and lactic acid and their salts. If desired, preservatives are typically present in the additives in amounts of from 0.001 to 2% by weight, based on the total weight of the additives.

Typical lipophilic dispersants are ascorbyl palmitate, polyglycerol fatty acid esters such as polyglycerol-3 polyricinoleate (PGPR90), sorbitan fatty acid esters, in particular sorbitan C₁₀-C₂₈-fatty acid esters such as, for example, mono- and di-C₁₀-C₂₈-fatty acid esters of sorbitan, such as sorbitan monolaurate, sorbitan monooleate and sorbitan monostearate (SPAN60), ethoxylated sorbitan fatty acid esters such as PEG(20) sorbitol monooleate, monoesters of lactic acid with saturated C₁₀-C₂₄-fatty acids, sugar esters of saturated C₁₆-C₂₀-fatty acids, propylene glycol fatty acid esters, and phospholipids such as lecithin. If desired, lipophilic dispersants are typically present in the additives in amounts of from 0.01 to 10% by weight, based on the total weight of the additives.

Protective colloids are polymeric substances which are suitable for dispersing water-insoluble solids in aqueous compositions, Suitable protective colloids which are approved for foodstuffs and feedstuffs comprise, for example,
1) proteinaceous protective colloids,
2) lignosulfonic acids, their salts, in particular their sodium salts and potassium salts,
3) oligo- and polysaccharides, including modified polysaccharides, such as, for example, dextrins, cellulose and cellulose derivatives such as methylcellulose, carboxymethylcellulose and their salts, hydroxyethylcellulose, hydroxypropylcellulose and hydroxypropylmethylcellulose, gum arabic, pectins,
4) polyvinyl alcohol, including partially hydrolyzed polyvinyl alcohol,
5) polyvinylpyrrolidone and mixtures of the abovementioned protective colloids.

The proteinaceous protective colloids of group 1) are a polymeric substance which is soluble or swellable in water and which is composed of amino acids and optionally includes glycosidic compounds. As a rule, the proteinaceous protective colloid is of vegetable or animal origin. Examples of proteinaceous protective colloids of group 1) are caseins and caseinates, including asi, ase, 1 and K casein and their mixtures, prolamins, i.e. storage proteins from cereal seeds such as gliadin, secalin, avenalin, hordein, zein, oryzin and kafirin, whey proteins such as beta-lactoglobulin, alpha-lactalbumin, serum albumin, proteose peptone, immunoglobulins and their mixtures, gelatin such as bovine, porcine and fish gelatin, in particular gelatin obtained by acid degradation, such as gelatin A100 and A200, gelatin obtained by alkaline degradation, such as gelatin B100 and B200, and gelatin types which have been obtained by enzymatic degradation, such as those products which are obtainable under the trademarks Gelita® Collage! A, Gelita® Sol DA, Gelita® Sol PA and Gelita® Sol LDA (DGF Stoess), furthermore soya protein and partially hydrolyzed soya protein and lupin protein. Lupin protein is understood as meaning plant proteins obtained from lupins. In accordance with a preferred embodiment, the pulverulent composition comprises at least one proteinaceous protective colloid from group 1), optionally in combination with an oligo- or polysaccharide from group 3). The molecular weight (weight average) of the proteinaceous protective colloid is typically in the range of from 10 000 to 250 000 Daltons, in particular in range of from 15 000 to 200 000 Daltons and specifically in the range of from 20 000 to 180 000 Daltons. Preferred protective colloids are casein, caseinates, for example sodium caseinate, soya protein, partially hydrolyzed soya protein, prolamin and lupin protein.

In accordance with a preferred group of embodiments of the invention, the food or feed additives are a solution or suspension of the astaxanthin composition according to the invention in an oil or fat which is suitable for foodstuffs or feedstuffs. The astaxanthin concentration in such additive formulations is, as a rule, in the range of from 0.01 to 30% by weight, in particular in the range of from 0.1 to 10% by weight, based on the total weight of the additive. In the case of a solution, the concentration is preferably in the range of from 0.01 to 3% by weight, in particular in the range of from 0.1 to 2% by weight. In the case of a suspension, the astaxanthin concentration is in the range of from 1 to 30% by weight, in particular in the range of from 2 to 20% by weight. The astaxanthin concentration refers to the total astaxanthin concentration in the form of the monoester, the diester which is optionally present and the nonesterified astaxanthin. Besides the oil which is suitable for foodstuffs or feedstuffs, the solution may comprise the abovementioned additives such as lipophilic dispersants and/or antioxidants and/or colorants, preferably in the abovementioned amounts. In the case of suspensions of the astaxanthin composition according to the invention, at least 80% by weight, in particular at least 90% by weight, of the particles of the astaxanthin composition have, as a rule, a particle diameter of below 100 μm, in particular of below 70 μm and specifically of below 50 μm. Frequently, the particles of the astaxanthin composition in the suspension have a volume-average particle diameter (D_{4,3} value) in the range of from 0.2 to 50 μm, in particular in the range of from 0.3 to 30 μm and specifically in the range of from 0.5 to 20 μm. The volume-average particle diameter is understood as meaning the volume-average particle diameter as determined by Fraunhofer diffraction of a dilute 0.01 to 0.1% by weight, specifically 0.05% by weight suspension.

The additives according to the invention for foodstuffs and feedstuffs may also be powders. As a rule, these powders also include other powder constituents, besides the astaxanthin composition according to the invention. These include, in particular, flow agents and fillers.

A flow agent, also referred to as antiagglomeration agent, is understood as meaning a solid, pulverulent substance which reduces the adhering of the powder particles to each other or to container walls. Flow agents are typically inert, solid, inorganic, pulverulent materials which are suitable for foodstuffs, for example oxides such as alumina, silica, in particular in the form of finely divided silica such as pyrogenic silica or silica gel (E 551), or titanium dioxide (E 171), phosphates such as tricalcium phosphate, hydroxides such as aluminum hydroxide, carbonates and hydrogen carbonates such as sodium carbonate (E 500), sodium hydrogen carbonate, potassium carbonate (E 501), magnesium carbonate (E 504) and calcium carbonate (E 170), or silicates such as sodium silicate (E 550), magnesium silicate (E 553a), talc (E 553b), calcium silicate (E 552), zinc silicate (E 557), aluminum silicates such as sodium aluminum silicate (E554), potassium aluminum silicate (E 555), calcium aluminum silicate (E 556), bentonite (E 558), kaolin (E 559), sodium chloride, and/or inert, solid, organic, pulverulent materials which are suitable for foodstuffs, such as, for example, crystalline mono-, di- and higher polysaccharides such as lactose, sugar (sucrose), cellulose powder, stearic acid, or stearates such as magnesium stearate (E 572), ammonium stearate (E 571) and aluminum stearate (E 573). Preferably, the flow agents have particle sizes which are below the particle size of the powder particles. Preferably, the particles of the flow agent have volume-average particle diameters in the range of from 1/100 to 1/2 of the volume-average particle diameter of the powder particles of the astaxanthin derivative. The amount of the flow agent will, as a rule, not exceed 10% by weight and, if present, typically accounts for 0.1 to 10% by weight, preferably 0.2 to 5% by weight, in particular 0.3 to 3% by weight, based on the total weight of the pulverulent additive.

The pulverulent additive compositions comprise, besides the astaxanthin composition, one or more further powder components. Besides the abovementioned flow agents, these include, in particular, other organic and inorganic substances which are suitable for foodstuffs and which are a solid at preferably 50° C., in particular 80° C. These include in particular the abovementioned fillers, protective colloids, antioxidants and preservatives.

As a rule, at least 90% by weight of the powder particles in the pulverulent compositions according to the invention have a particle diameter of below 500 μm, in particular not more than 200 μm. As a rule, the powder particles have a mean particle size (particle diameter, volume average, D4,3 value) in the range of from 0.5 to 300 μm, especially in the range of from 0.5 to 200 μm.

In one group of embodiments of pulverulent food and feed additives according to the invention, the pulverulent formulation comprises at least one protective colloid. These compositions are dispersible in water. The weight ratio of protective colloid to the astaxanthin components of the astaxanthin composition is typically in the range of from 50:1 to 1:5, in particular in the range of from 20:1 to 1:2 and specifically in the range of from 10:1 to 1:1.

In preferred embodiments of pulverulent food and feed additives, the pulverulent formulation comprises at least one proteinaceous protective colloid. In accordance with a first group, the proteinaceous protective colloid is in particular a soya protein or a mixture of soya protein and a further proteinaceous protective colloid, for example a mixture of soya protein and a prolamin, a mixture of soya protein and a lupin protein, a mixture of soya protein and a casein and/or caseinate, a mixture of soya protein and soya protein hydrolysate. In particular, the soya protein forms the main component of the proteinaceous protective colloid; its content in the protective colloid amounts to at least 50% by weight, in particular at least 80% by weight or at least 90% by weight. In accordance with a second group, the proteinaceous protective colloid is in particular a partially hydrolyzed soya protein or a mixture of partially hydrolyzed soya protein and a further proteinaceous protective colloid, for example a mixture of partially hydrolyzed soya protein and a prolamin, a mixture of partially hydrolyzed soya protein and a lupin protein, a mixture of partially hydrolyzed soya protein and a casein and/or caseinate. In particular, the partially hydrolyzed soya protein forms the main component of the proteinaceous protective colloid; its content in the protective colloid amounts to at least 50% by weight, in particular at least 80% by weight or at least 90% by weight. A partially hydrolyzed soya protein is understood as meaning a soya protein which has been subjected to partial enzymatic degradation with a protease. The degree of hydrolysis DH of such a partially hydrolyzed soya protein is, as a rule, at least 1%, in particular at least 5%, for example in the range of from 1 to 20% and in particular in the range of from 5 to 16%. The degree of hydrolysis DH is understood as meaning the quotient of the number of hydrolyzed peptide bonds to the total number of peptide bonds in the original protein. The degree of hydrolysis can be determined by what is known as the “pH-stat method” as described by C. F. Jacobsen et al. in “Methods of Biochemical Analysis”, Vol. IV, P. 171-210, Interscience Publishers Inc., New York 1957. Soya proteins which are employed for the preparation of the formulations according to the invention, but also for the preparation of the partially hydrolyzed soya proteins, are usually commercially available soya protein isolates and concentrates with protein contents of, as a rule, 70 to 90% by weight, with the remaining 10 to 30% by weight being more or less undefined other plant constituents.

In preferred embodiments of pulverulent food and feed additives, the pulverulent formulation comprises at least one protective colloid from the group of the lignosulfonates, in particular sodium lignosulfonate, optionally in combination with further protective colloids, in particular those from group 3). In this embodiment, it is in particular the lignosulfonate which forms the main component of the protective colloid; its content in the protective colloid amounts to at least 50% by weight, in particular at least 80% by weight or at least 90% by weight.

The invention also relates to foodstuffs which comprise an astaxanthin composition according to the invention, where the concentration of the astaxanthin composition corresponds to the astaxanthin content in the foodstuff which is approved for foodstuffs. Typically, the concentration of the composition according to the invention, calculated as astaxanthin, is in the range of from 1 to 500 mg/kg of the respective foodstuff.

Examples of foodstuffs which have had the compositions according to the invention added to them in accordance with the invention comprise fatty food such as sausage, cheese, edible oils, weight gainers, protein food, power bars, functional drinks such as sports and wellness drinks and high-calorie drinks, astronaut food, tube feeds and the like.

The invention also relates to feedstuffs which comprise an astaxanthin composition according to the invention, where the concentration of the astaxanthin composition corresponds to the astaxanthin content in the feedstuff which is approved for the respective feedstuff. Typically, the concentration of the composition according to the invention, calculated as astaxanthin, is in the range of from 1 to 500 mg/kg of the respective feedstuff.

Typical components in feedstuffs are carbohydrate sources, in particular cereal meals such as wheat or corn meal, soybean meal, but also sugar and sugar alcohols, furthermore proteinaceous components such as soya concentrate, fish meal, glutens such as corn or wheat gluten, oils and fats, for example the abovementioned edible oils, but also other edible fats of vegetable or animal origin, furthermore nutraceuticals such as free amino acids, their salts, vitamins and trace elements, and optionally processing aids, for example glidants, anticaking agents, inert fillers and the like, and optionally preservatives. Typical fish feed compositions comprise, for example, cereal meal in an amount of, for example, from 3 to 20% by weight, gluten, for example in amount of from 1 to 30% by weight, one or more protein sources, for example soya concentrate and/or fish meal, for example in a total amount of from 10 to 50% by weight, fats and/or oils in an amount of, for example, from 10 to 50% by weight, optionally one or more vitamins in a total amount of, for example, from 0.1 to 2% by weight and optionally amino acids in an amount of, for example, from 0.1 to 5% by weight, in each case based on the total amount of the feedstuff components.

The foodstuffs or feedstuffs are successfully prepared by adding the desired amount of a food or feed additive according to the invention to the foodstuff. The addition of the additive may take place during the manufacturing of the foodstuff or feedstuff, or by addition to the foodstuff or feedstuff components when making the foodstuff or feedstuff.

A specific embodiment of these feedstuffs relates to feedstuff pellets, specifically feedstuff pellets for fish feed, which are loaded with the astaxanthin derivative solution which is obtainable in accordance with the invention. Such pellets typically comprise the astaxanthin composition according to the invention in an amount of from 10 to 200 ppm, based on the total weight of the feedstuff and calculated as astaxanthin. As a rule, such pellets are prepared by spraying traditional pellets with a solution of the astaxanthin composition according to the invention, preferably under reduced pressure, where the spraying may be carried out continuously or, preferably, batchwise. For example, it is possible to introduce the traditional pellets into a suitable container, to apply a vacuum to the container and then to spray on a solution of the astaxanthin composition in an oil suitable for foodstuffs, while mixing the pellets, and subsequently to introduce air into the container. A uniform penetration of the oil solution into the pellets is achieved in this manner. Optionally, it is possible to reapply a vacuum and to spray on again the solution of the astaxanthin composition in an oil which is suitable for foodstuffs in the above-described manner. Pellets which comprise the oil and the astaxanthin composition according to the invention in the core are obtained in this manner.

The invention also relates to food supplements. These food supplements comprise the astaxanthin composition according to the invention in formulated form, i.e. they comprise, besides the astaxanthin composition according to the invention, at least one adjuvant which is approved for food supplements. These include mainly the adjuvants mentioned in connection with the food additives, such as carriers, dispersants, antioxidants, preservatives and colorants, and other adjuvants which are required for preparing the desired form of administration.

Typical forms of administration for food supplements are pulverulent forms, powders, granules, tablets, in particular film-coated tablets, lozenges, sachets, cachets, sugar-coated tablets, capsules such as hard- and soft-gelatin capsules, effervescent tablets and effervescent powders.

The amount of astaxanthin composition in the food supplement is typically in the range of from 0.5 to 100 mg, in particular 1 to 50 mg, per unit dose, calculated as astaxanthin.

Subject matter of the invention are the astaxanthin compositions described herein for the therapeutic use as medicament and as constituent for a medicinal preparation (=pharmaceutical composition). Suitable indications in which the compositions according to the invention can be employed are the indications mentioned above.

The use according to the invention of the compounds described comprises, within the scope of the treatment, a method. Here, an effective amount of the astaxanthin composition according to the invention, as a rule formulated according to pharmaceutical and veterinary practice, is administered to the individual to be treated, preferably to a mammal, in particular a human, farm animal or domestic animal. Whether such a treatment is indicated and in which form it should be carried out depends on the individual case and requires a medical assessment (diagnosis) which takes into consideration existing signs, symptoms and/or malfunctions, risks of developing certain signs, symptoms and/or malfunctions, and other factors.

As a rule, the treatment is carried out by one or more administration(s) per day, optionally together or alternately with other active substances or active-substance-comprising preparations, so that a daily dose of preferably approximately 0.1 to 50 mg/kg body weight, in particular 0.5 to 10 mg/kg body weight, or 1 to 1000 mg, in particular 2 to 200 mg, in each case calculated as astaxanthin, is administered to an individual to be treated, preferably by oral administration.

The invention also relates to the manufacture of medicinal preparations, hereinbelow also referred to as pharmaceutical compositions, for the treatment of an individual, preferably a mammal, in particular a human, farm animal or domestic animal. Thus, the astaxanthin compositions according to the invention are usually administered in the form of medicinal preparations which comprise a pharmaceutically acceptable adjuvant, i.e. an excipient, the astaxanthin composition according to the invention and optionally one or more further active substances. These preparations are preferably administered via the oral route.

Examples of suitable medicinal preparations comprise solid pharmaceutical forms, such as pulverulent forms, powders, granules, tablets, in particular film-coated tablets, lozenges, sachets, cachets, sugar-coated tablets, capsules such as hard- and soft-gelatin capsules, effervescent tablets, effervescent powders, semi-solid pharmaceutical forms such as ointments, creams, hydrogels, pastes or patches, and liquid pharmaceutical forms such as solutions, emulsions, in particular oil-in-water emulsions, suspensions, for example lotions, eye drops and eardrops. Liposomes or microcapsules may furthermore also be used.

When manufacturing the medicinal preparations, astaxanthin compositions according to the invention are usually mixed or diluted with an excipient. Excipients may be solid, semi-solid or liquid materials which act as vehicles, carriers or medium for the active substance. The compositions according to the invention typically comprise the astaxanthin composition in an amount which corresponds to the daily dose or to a fraction of the daily dose, for example, to half, a third of, or a quarter of the daily dose. The amount of astaxanthin composition in the medicinal preparation is typically in the range of from 0.5 to 200 mg, in particular 1 to 100 mg, per unit dose, calculated as astaxanthin.

Suitable excipients are listed in the relevant medicinal monographs. Furthermore, the formulations may comprise pharmaceutically acceptable carriers or customary adjuvants, such as glidants; wetting agents; emulsifiers and suspending agents; preservatives; antioxidants; antiirritants; chelating agents; coating auxiliaries; emulsion stabilizers; film formers; gel formers; odor masking agents; taste corrigents; resins; hydrocolloids; solvents; solubilizers; neutralizing agents; diffusion accelerators; pigments; quaternary ammonium compounds; refatting- and superfatting agents; raw materials for ointments, creams or oils; silicone derivatives; spreading auxiliaries; stabilizers; sterilants; suppository bases; tablet adjuvants, such as binders, fillers, glidants, disintegrants or coatings; propellants; drying agents; opacifiers; thickeners; waxes; plasticizers and white mineral oils. A formulation in this regard is based on specialist knowledge as described, for example, in Fiedler, H. P., Lexikon der Hilfsstoffe für Pharmazie, Kosmetik and angrenzende Gebiete [Encyclopedia of adjuvants for Pharmacy, Cosmetics and Related Fields], 4th edition, Aulendorf: ECV-Editio-Kantor-Verlag, 1996.

For the following studies, the following astaxanthin compositions were employed. The term area % stands for the area percentage as determined by HPLC:

Astaxanthin monooleate with the following properties:

astaxanthin monooleate content>91% by weight;

E/Z isomer distribution in the astaxanthin skeleton>45 area % all-E isomer;

enantiomeric purity: >95% ee 3S, 3′S enantiomer;

trans-fatty acid ester content>40% by weight.

Astaxanthin dioleate with the following properties:

astaxanthin dioleate content>93% by weight;

E/Z distribution in the astaxanthin skeleton>60 area % all-E isomer;

enantiomeric purity: >95% ee 3S, 3′S enantiomer;

trans-fatty acid ester content 60% by weight.

Natural astaxanthin product from mixture of mono- and diesters of astaxanthin (predominantly 3S, 3′S enantiomer) with C₁₆-C₂₂-fatty acids from Haematococcus pluvialis with the following composition (as determined by HPLC-MS, data in area %)

1.6% astaxanthin-C18:4-monoester

9.7% astaxanthin-C18:3-monoester

15.0% astaxanthin-C18:2-monoester

28.7% astaxanthin-C18:1-monoester

5.3% astaxanthin-C18:0-monoester

The remaining 39.7% is shared by 77 unidentified peaks with in each case <2 area %, which, however, are predominantly mono- and diesters of astaxanthin.

### PREPARATION EXAMPLE 1

### Preparation of Astaxanthin Monopalmitate

3 g (11.7 mmol) palmitic acid were introduced into 47.37 ml (53 g, 740 mmol) dichloromethane. 2.85 g (17.55 mmol) 1,1′-carbonyldiimidazole (CDI) were added in three portions at room temperature in intervals of in each case 5 minutes. Stirring was continued overnight, and 3.49 g (5.85 mmol) of astaxanthin (3S, 3′S enantiomer, ee>95%) were added on the next day. After 6 hours, 133.8 μl of acetic acid were added to the reaction mixture, and stirring was continued overnight at room temperature. After 20 hours, one sample was examined by thin-layer chromatography (silica gel, mobile phase cyclohexane/ethyl acetate=1:2). The thin-layer chromatogram showed that part of the astaxanthin employed had been converted into astaxanthin monopalmitate. Only very small amounts of astaxanthin dipalmitate were detected.

The astaxanthin monopalmitate can be separated from the reagents and the unreacted astaxanthin by chromatographing the reaction mixture on silica gel using cyclohexane/ethyl acetate in a volume ratio of 1:2. In this manner, astaxanthin monopalmitate with a purity of >80% by weight, in particular >90% by weight, can be obtained.

Astaxanthin monooleate in a purity of >80% by weight, in particular >90% by weight, can be obtained analogously.

Bioavailability Studies

The bioavailability was studied by determining the blood plasma level of astaxanthin in G\ddot{p}ttinger minipigs. The integral plasma concentration in the blood serum over 96 h (AUC) was determined as a measure for the bioavailability.

All astaxanthin derivatives were administered as solutions in olive oil:

astaxanthin monooleate, 4.2% by weight (calculated as free astaxanthin) in olive oil formulation 1:

astaxanthin dioleate, 3.1% by weight (calculated as free astaxanthin) in olive oil formulation 2:

natural astaxanthin product, 2.4% by weight (calculated as free astaxanthin) in olive oil formulation 3:

The study was carried out on three 4-5 months old male Göttinger minipigs with a body weight of 9.5-10.5 kg. For taking blood samples, a catheter was implanted into the femoral vein of all minipigs. Two of the minipigs (pig 2 and pig 3) additionally had a catheter implanted into the jugular vein. During the study period, the catheters were flushed daily with physiological saline.

During the non-treatment times, the minipigs were fed a commercial ration for minipigs (by SDS, Essex, UK). The ration was fed in the morning and in the afternoon on week days and in the early morning and just before midday over the weekend.

On the day of treatment, a high-fat ration which had been fortified with 20% of corn oil was fed. To this end, 224 g of the commercial minipig ration were mixed with 54 g of corn oil. Pig 1 received 140 g of the high-fat diet immediately before treatments 1 and 2 and 140 g immediately after treatments 1 and 2 and 140 g 2 h before treatment 3 and 140 g 1 h after treatment 3. Pigs 2 and 3 received in each case 170 g of the high-fat ration immediately before treatments 1 and 2 and 110 g immediately after treatments 1 and 2 and 170 g 2 h before treatment 3 and 110 g 1 h after the third treatment.

The treatments were each separated by a period of 1 week. The treatment regimen was as shown in Table 1:

TABLE 1
Treatment regimen

| | Treatment 1 | Treatment 2 | Treatment 3 |
|---|---|---|---|
| Pig 1 | formulation 3 | formulation 2 | formulation 1 |
| Pig 2 | formulation 1 | formulation 3 | formulation 2 |
| Pig 3 | formulation 2 | formulation 1 | formulation 3 |

For the treatment, the formulation was administered, as a single dose, to the respective pig using a gastric tube. The single dose was 50 mg/kg astaxanthin. Immediately before the administration and 2 h, 6 h, 7 h, 8 h, 9 h, 10 h, 11 h, 12 h, 1 h, 24 h, 32 h, 48 h, 56 h, 72 h and 96 h after the administration, approximately 2 ml of blood were sampled from the pigs via the femoral vein. The samples were stored on ice and, within 30 min after sampling, centrifuged for 10 min at 1500 g and 4-8° C. The plasma was transferred into sterile sealable polypropylene containers (Micronic, Lelystadt) and stored at temperatures below −75° C. until analysis. The astaxanthin level in the blood plasma was determined by HPLC at WIL Research, Den Bosch NL. The results are shown in Table 2 hereinbelow. The data shown are the means for the three test pigs.

TABLE 2

| | | | |
|---|---|---|---|
| tₗₐₛₜ [h] | 48-56 | 48-56 | 24-23 |
| tₘₐₓ [h] | 6-12 | 8-11 | 6-9 |
| Cₘₐₓ [ng/mL] | 61.8 | 89.9 | 5.54 |
| Cₗₐₛₜ [ng/mL] | 2.83 | 6.38 | 1.71 |
| AUCₗₐₛₜ [h · ng/mL] | 941 | 1560 | 65.6 |
| AUC_{∞} [h · ng/mL] | 996 | 1670 | 118 |
| t_{1/2} [h] | 10.3 | 9.95 | 18.9 |

tₗₐₛₜ = period until the values fell short of the detection level
tₘₐₓ = period of maximum serum level
Cₘₐₓ = maximum serum level
Cₗₐₛₜ = concentration before the values fell short of the detection level
AUClast = concentration integral up to the point in time before the values fell short of the detection level
AUC_{∞} = extrapolated concentration integral
t_{1/2} = half life

1.-18. (canceled)

19. The use of astaxanthin compositions in foodstuffs, food supplements or feedstuffs for non-human mammals, where the astaxanthin composition comprises at least 90% by weight, based on the total weight of astaxanthin and astaxanthin derivatives in the composition, of a monoester of astaxanthin with an aliphatic C₁₀-C₂₂-monocarboxylic acid, where at least 90% by weight of the monoester present in the astaxanthin composition is a monoester with precisely one aliphatic, unbranched C₁₆-C₂₂-monocarboxylic acid.

20. The use according to claim 19, wherein at least 90% by weight of the astaxanthin monoester is a monoester with an at least monounsaturated, aliphatic, unbranched C₁₆-C₂₂-monocarboxylic acid, including a monoester with a cis/trans mixture and mixtures of the double-bond isomers of this C₁₆-C₂₂-monocarboxylic acid.

21. The use according to claim 20, wherein the at least monounsaturated fatty acid is selected among C18-1, C18-2, C18-3 and C18-4 fatty acids.

22. The use according to claim 21, wherein at least 90% by weight of the astaxanthin monoester is the monoester with a C18-1 fatty acid.

23. The use according to claim 20, wherein less than 60% by weight of the unsaturated fatty acid which is present in the astaxanthin monoester are present as trans-fatty acid.

24. The use according to claim 19, wherein at least 40% by weight of the astaxanthin monoester are present in the form of a monoester of all-trans astaxanthin.

25. The use according to claim 19, wherein at least 90% by weight of the astaxanthin monoester are present in the form of a monoester of an astaxanthin which either has the (S) or the (R) configuration in the asymmetric centers in positions 3 and 3′, respectively.

26. An astaxanthin composition in the form of a food additive, feed additive or food supplement, comprising an adjuvant which is suitable for foodstuffs, feedstuffs or food supplements, where the astaxanthin composition comprises at least 90% by weight, based on the total weight of astaxanthin and astaxanthin derivatives in the composition, of a monoester of astaxanthin with an aliphatic C₁₀-C₂₂-monocarboxylic acid, where at least 90% by weight of the monoester present in the astaxanthin composition is a monoester with precisely one aliphatic, unbranched C₁₆C₂₂-monocarboxylic acid.

27. The composition according to claim 26, selected among solutions and suspensions of the astaxanthin composition and an oil or fat which is suitable for foodstuffs or feedstuffs.

28. The composition according to claim 26 in the form of a powder, comprising an astaxanthin composition and a protective colloid.

29. The composition according to claim 26 in the form of a food supplement for oral administration.

30. A process for the preparation of a foodstuff or feedstuff, comprising adding an astaxanthin composition according to claim 26 to a conventional foodstuff or feedstuff.

31. An astaxanthin composition for the therapeutic use as medicament and as constituent for a medicinal preparation, where the astaxanthin composition comprises at least 90% by weight, based on the total weight of astaxanthin and astaxanthin derivatives in the composition, of a monoester of astaxanthin with an aliphatic C₁₀-C₂₂-monocarboxylic acid, where at least 90% by weight of the monoester present in the astaxanthin composition is a monoester with precisely one aliphatic, unbranched C₆C₂₂-monocarboxylic acid.

32. The astaxanthin composition according to claim 31 for the treatment or prevention of joint diseases, eye diseases, skin diseases, skin aging, heart diseases, diseases of the blood vessels, inflammatory diseases, tumor diseases, bacterial infections, type-2 diabetes and associated sequelae, and degenerative nerve disorders, for improving male fertility, memory, physical fitness and concentration and for strengthening the immune system.

33. A pharmaceutical astaxanthin composition comprising at least one pharmaceutically suitable auxiliary, where the astaxanthin composition comprises at least 90% by weight, based on the total weight of astaxanthin and astaxanthin derivatives in the composition, of a monoester of astaxanthin with an aliphatic C₁₀-C₂₂-monocarboxylic acid, where at least 90% by weight of the monoester present in the astaxanthin composition is a monoester with precisely one aliphatic, unbranched C₁₆-C₂₂-monocarboxylic acid.
